(19) United States
(12) Patent Application Publication
(10) Pub. No.: US 2014/0025099 A1
(43) Pub. Date: Jan. 23, 2014

(54) Adjustable Ergonomic Grip

(71) Applicant: Richard Morrison, (US)

(72) Inventor: Richard Morrison, (US)

(21) Appl. No.: 13/553,282

(22) Filed: Jul. 19, 2012

(51) Int. Cl. A61M 37/00

(52) U.S. Cl. CPC A61M 37/0076
USPC 606/186

(57) ABSTRACT

The preferred embodiment of the present invention comprises a grip for a hand held instrument such as a tattoo tube designed to ergonomically enhance the finger and thumb placement, which grip is adjustable and therefore customizable by the user and characterized by a housing featuring a series of circumferential grooves spaced along the exterior of the housing and removable flexible o-rings adjustably situated within the grooves such that the user's fingers and thumb can comfortably and safely grip the tattoo tube with less pressure and reduce repetitive stress injuries.

### CROSS-REFERENCES TO RELATED APPLICATIONS—Not Applicable

### STATEMENT REGARDING FEDERALLY SPONSORED RESEARCH OR DEVELOPMENT—Not Applicable

### PARTIES TO A JOINT RESEARCH AGREEMENT—Not Applicable

### BACKGROUND

Tattooing is performed by means of a sharp, small diameter needle or cluster of needles, dipped in or otherwise supplied with a pigment or ink which is then inserted into the epidermal layer of the skin. Insertion occurs via vibration or other means of repeated injection. The needle is carried in a tubular holder, often referred to in the industry as a tattoo tube, which is attached to a tattoo machine which provides the source of the vibration.

The constant vibration from the tattoo machine can cause early fatigue in the tattoo artist's hands and fingers. Both the fatigue and the constant vibration can also make the tattoo tube difficult to grasp and hold for significant time periods. The preferred embodiment of this invention described herein solves both of those problems due to its innovative design and adjustable ergonomic features.

The preferred embodiment of this invention comprises two basic components. The first component is a housing. generally in the form of an elongated tube characterized by an exterior profile exhibiting a series of circumferential depressions.

The second component of the preferred embodiment consists of a plurality of flexible o-rings that fit snugly into the depressions such that the o-rings can be adjusted along the housing in various positions along the length of the housing. Spaces between the o-rings allow for the tattoo artist to maintain sensitivity with the base as well as to secure his fingers and thumb in position on the tattoo tube.

Prior art has not resolved these deficiencies. In Ferranti, U.S. Pat. No. 6,390,818, the invention teaches a pliant elastomeric sleeve adapted to fit the shank of a dental tool with the purpose of ergonomically enhancing the finger grip. Unlike the present invention, however, it does not allow for adjustability of the finger and thumb along the shaft as do the o-rings of the present invention nor does its elongated uninterrupted continuous surface allow for specific placement of the finger and thumb for purposes of increasing stability as do the grooves and o-rings of the present invention.

In Yanagisako, U.S. Pat. No. 7,241,064, and in Schwartz, U.S. Pat. No. 4,601,598, the invention presents a grip for a writing utensil comprised of a plurality of deformable flange-like projections in series along the shaft. However, the invention is universal for all users as the projections are not adjustable for each individual tattoo artist.

For the foregoing reasons there remains a need for an adjustable ergonomic grip for a tool handle such as that utilized in a tattoo tube.

It is accordingly the general object of the present invention to provide an ergonomic grip for tattoo tubes and other similar tools, such as dental instruments, to lessen fatigue from use and avoid injuries. It is a further object of the present invention to provide a more secure and thus safer grip that it is customizable for the individual user.

### SUMMARY

The preferred embodiment of the present invention is directed toward providing an adjustable ergonomic grip for instruments, such as tattoo tubes, which are further customizable for the user. Particularly it relates to a grip to prevent fatigue and possibly neurological disorders that may stern from the constant vibration of such a tool while in use. The preferred embodiment comprises an elongated shaft or tube characterized by a series of exterior circumferential depressions fitted with flexible o-rings that may be adjusted or removed for the user's comfort and control. By providing the cushioning from the o-rings, and the ability to move and adjust the o-rings for each individual user's thumb and finger specific conformation and comfort, the user can work more efficiently and safely.

### BRIEF DESCRIPTION OF THE DRAWINGS

These and other features and advantages of the present invention will become better understood with regard to the following descriptions, appended claims and accompanying drawings as follows:

FIG. 1 is an outline of the of the invention with the o-rings removed.

FIG. 2 is a side view of the exterior of the invention with the o-rings removed.

FIG. 3 is a sectional view of the invention with the o-rings removed along the lines A-A of FIG. 2.

FIG. 4 is a perspective view of one embodiment of the invention with the o-rings removed.

FIG. 5 is a plan view illustrating the relationship between the depressions and the spaces characterized by the exterior grooves.

FIG. 6 is a top view of the o-ring.

FIG. 7 is a sectional view of the o-ring along the lines B-B of Fig.

FIG. 8 is a sectional view of the invention including the o-rings along the lines A-A of FIG. 2 and also illustrating one embodiment of the interior of the housing.

FIG. 9 is a perspective view of the invention.

FIG. 10 is a side view of the invention.

FIG. 11 is an end view of the invention.

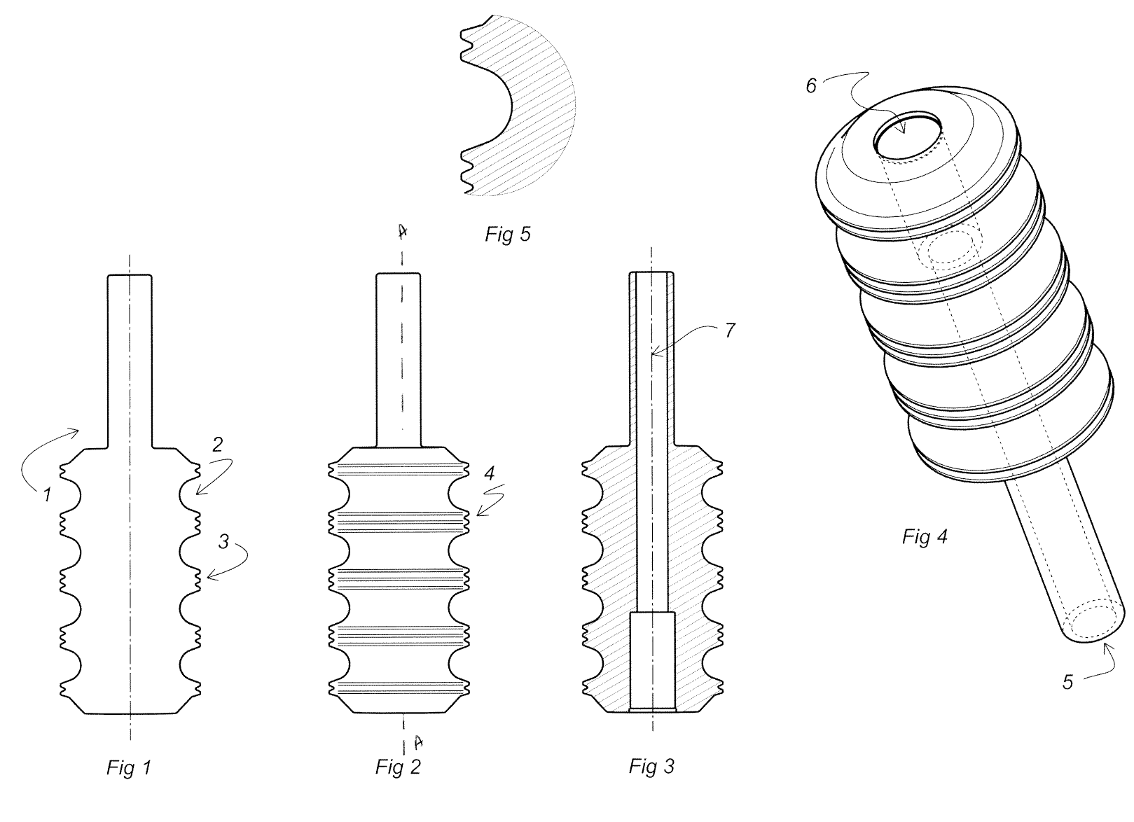

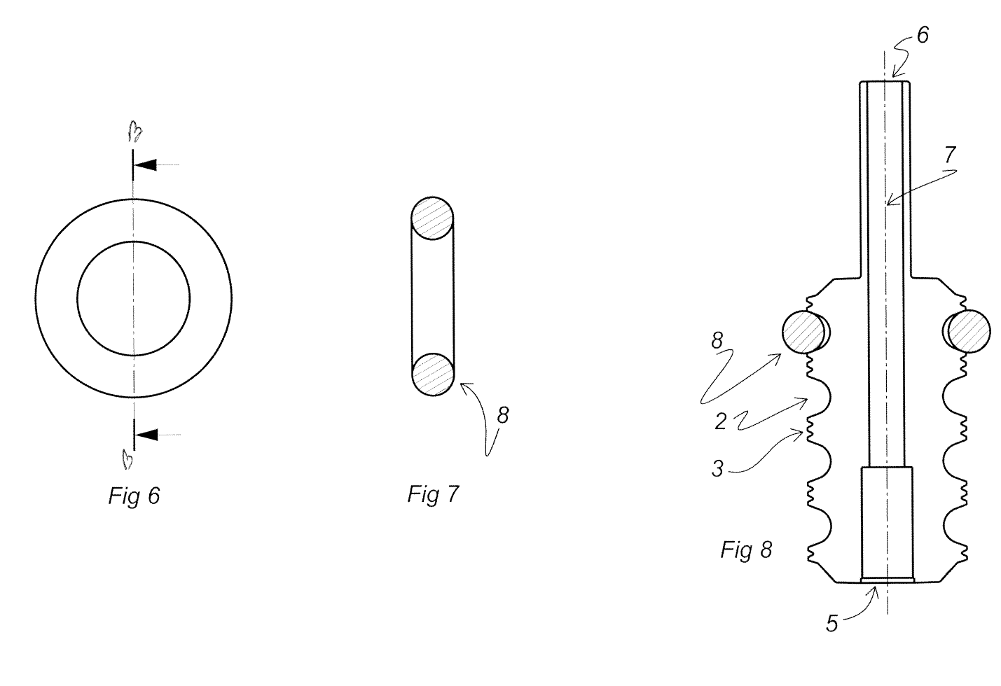

### DETAILED DESCRIPTION

Before explaining at least one embodiment of the present invention in detail, it is to be understood that this invention is not limited in its application to the details of construction and to the arrangements of the components set forth in the following description or illustrated in the drawings. The invention is capable of other embodiments and of being practiced and carried out in various ways. Numerous details of construction may be varied through a wide range without departing from the principle of the invention. It is also to be understood that the terminology used herein is for the purpose of describing particular embodiments only, and is not intended to be limiting as the scope of the present invention will be limited only by the appended claims. Unless defined otherwise, all terms used herein have the same meaning as commonly understood by one of ordinary skill in the art to which this invention belongs.

Referring now to the drawings, the preferred embodiment of the present invention is illustrated in FIGS. 4 and 9 and consists of a housing 1, comprised of a back stem 5 that is adapted to attach to a tattoo machine and an opposing end 6 adapted to receive a tattoo needle. Alternatively, the opposing end 6 may consist of a shaped tip to hold the tattoo needle. The shaped tip can be round, diamond shaped or any other shape customarily used in the tattoo industry. The housing 1 may be any shape although a cylindrical housing, or elongated tube. is preferred with a further preferred diameter of about one inch for the portion of the housing 1 between the back stem 5 and opposing end 6.

The tattoo tube may be comprised of a hollow housing 1 with open end 6 to receive the tattoo needle and back stem 5 adapted for attachment to a tattooing device, or may be comprised of an internal longitudinal opening between back stem 5 and open end 6 as depicted in FIG. 3. The open end 5 may be of the same diameter as a separate shaped tip, preferably of surgical steel, such that the shaped tip is held in place by the convergent force of the open end 5 of the housing 1 against the shaped tip. Alternatively, the shaped tip may be attached to the open end 5 by other means such as an adhesive.

The preferred length of the portion between the back stem 5 and opposing end 6 is about two inches. The housing 1 may be made from any suitable material but is preferably made from stainless steel or bronze or in the case of disposable tattoo tubes, a thermoplastic material such as acrylonitrile butadiene styrene (ABS plastic).

The housing 1 includes an exterior profile characterized by a plurality of circumferential depressions 2, preferably four, equally spaced along the length of the housing between the back stem and shaped tip. Preferably each depression is about ¼ inch wide and ⅛ inch deep. Alternatively each depression 2 may be separated by a space 3 which is further characterized by a series of circumferential grooves 4, preferably two, for further placement of and stabilizing the fingers. The preferred length of each space 3 is about ⅛ inch.

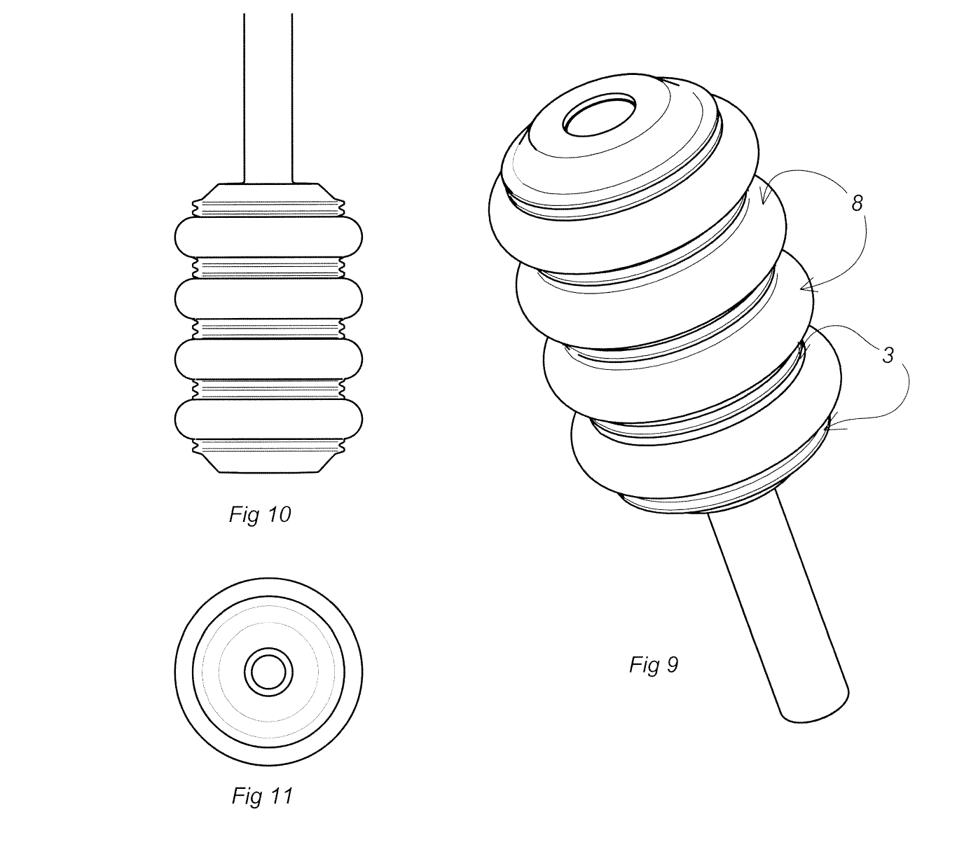

A plurality of flexible elastomeric o-rings 8 of such necessary internal diameter to fit snugly and remain stationery within the depressions 2, are detachably and flexibly affixed to the housing 1 and seated within the depressions 2 as depicted in FIG. 8 and FIG. 9 such that they can be arranged for maximum comfort, preferred finger and thumb placement, and prevention of digit slippage. Said o-rings 8 must be of sufficient exterior diameter to extend beyond the spaces 3 on the housing 1 such that they make contact with the user's fingers. Preferably, for a housing of approximately one inch diameter said o-rings are about ¼ inch thick with an external diameter of about 1¼ inch. Said o-rings may be made of any soft flexible material but are preferably made from ethylene-propylene or silicone.

The preferred embodiment of this invention provides greater control through the enhanced stability of the user's grip. By reducing the gripping effort, stress and fatigue are eliminated or greatly decreased, and safety and comfort is increased. The depressions 2 absorb the pressure from the fingers while the o-rings 8 stabilize the finger position and also absorb laterally induced vibratory pressure.

Alternatively, the grip described above can be utilized in any hand held instrument particularly those involving vibratory pressure such as dental drills.

The previous described versions of the present invention have many advantages including providing a disposable tattoo tube that provides comfort and stability to the tattoo artist which in turn allows for a safer and more efficient tattoo experience that is economical to manufacture.

It is to be understood that while certain forms of this invention have been illustrated and described, the invention is not limited thereto, except insofar as such limitations are included in the claims.

1. An adjustable ergonomic grip for a tattoo tube or similar handheld instrument comprise
a housing with two opposing ends;
said housing having an exterior profile characterized by a plurality of circumferential depressions spatially arranged in series along the exterior of the housing between said opposing ends;
a plurality of flexible o-rings each sized to fit snugly into the depressions on the housing, the outer diameter of the o-rings being larger than the outer diameter of the housing so that the o-rings extend above the housing and make contact with the user's fingers.

2. The grip according to claim 1 wherein the housing is comprised of a back stem adapted for attachment to a tattooing device, or other means of energizing the instrument, and an opposing end having an opening to allow passage of a tattoo needle;

3. The grip according to claim 2 wherein the opposing end is comprised of a shaped tip;

4. The grip according to claim 2 wherein the opposing end is of such a diameter as to enable the insertion of and retainage of a separate shaped tip whereby the shaped tip is held in place by the convergent force of the opposing end of the housing against the shaped tip;

5. The grip according to claim 2 wherein the shaped tip is fastened to the opposing end by an adhesive;

6. The grip according to claim 3 wherein the shaped tip is made of surgical steel.

7. The grip according to claim 1 wherein the housing is comprised of an elongated tube.

8. The grip according to claim 1 wherein the depressions are separated along the housing by a space;

9. The grip according to claim 2 wherein the portion of the housing between the back stem and opposing end is approximately one inch in diameter and approximately two inches in length.

10. The grip according to claim 8, wherein the spaces separating the depressions are characterized by a plurality of exteriorly positioned circumferential grooves to provide more stability for the fingers to grip the tattoo tube or other such instrument;

11. The grip according to claim 1, wherein the said housing is made of a thermoplastic material such as acrylonitrile butadiene styrene or other suitable material for use in disposable tattoo tubes;

12. The grip according to claim 1, wherein the said housing is made of a material capable of withstanding high heat for purposes of autoclaving;

13. The grip according to claim 1, where the said depressions are about ¼ inches wide and about ⅛ inch deep.

14. The grip according to claim 1, where the said o-rings are about ¼ inch thick and have an exterior diameter about 1¼ inches.

15. The grip according to claim 8 wherein the said spaces between the said depressions are about ⅛ inch wide.

16. A pliant elastomeric sleeve adapted to removeably fit over the elongate shaft of a tattoo tube or handle of other similar handheld instruments comprised of:
an axially extending generally cylindrical tube sized to fit snugly over the handle of the instrument;
said tube further comprised of an exterior profile characterized by a plurality of circumferential depressions spatially arranged in series along the tube;
a plurality of flexible o-rings that are sized to fit snugly into the depressions such that a portion of the o-rings sits above the depression in contact with the user's fingers.

17. The sleeve according to claim 16 wherein the depressions are separated by a space;

18. The sleeve according to claim 16 wherein the spaces between the depressions are characterized by a plurality of circumferential grooves.
